(19) United States
(12) Patent Application Publication
(10) Pub. No.: US 2014/0349974 A1
(43) Pub. Date: Nov. 27, 2014

(54) ZOLEDRONIC ACID DOSAGE FORMS FOR THE TREATMENT OF PAIN

(71) Applicant: Antecip Bioventures II LLC, New York, NY (US)

(72) Inventor: Herriot Tabuteau, New York, NY (US)

(21) Appl. No.: 14/457,659

(22) Filed: Aug. 12, 2014

(51) Int. Cl. A61K 31/197; A61K 31/675

(52) U.S. Cl. CPC A61K 31/197; A61K 31/675
USPC 514/94

(57) ABSTRACT

Treatment of pain and related conditions with oral dosage forms of zoledronic acid is described herein.

### SUMMARY

The present disclosure is directed towards treating medical conditions, such as pain, using new forms of zoledronic acid, which have the therapeutic efficacy of zoledronic acid discussed above, with improved aqueous solubility, rate of dissolution, and/or improved permeability and thus enhanced bioavailability. Treatment may be carried out using molecular complexes of zoledronic acid that includes cocrystals, salts, and solvates (e.g. hydrates and mixed solvates as well as solvates of salts), and mixtures containing such materials.

The disclosure further includes compositions of molecular complexes of zoledronic acid suitable for incorporation in a pharmaceutical dosage form for the treatment of various medical conditions. Specific molecular complexes pertaining to the disclosure include, but are not limited to, complexes of zoledronic acid with sodium, ammonium, ammonia, L-lysine, DL-lysine, nicotinamide, adenine, and glycine. Obvious variants of the disclosed zoledronic acid forms in the disclosure, including those described by the drawings and examples, will be readily apparent to the person of ordinary skill in the art having the present disclosure and such variants are considered to be a part of the current invention.

### DETAILED DESCRIPTION

An oral dosage form of a bisphosphonate described herein, such as zoledronic acid, may be used to treat, or provide relief of, any type of pain including, but not limited to, inflammatory pain, arthritis pain, complex regional pain syndrome, lumbosacral pain, musculoskeletal pain, neuropathic pain, chronic pain, cancer-related pain, acute pain, postoperative pain, etc. In some instances, pain relief may be palliative, or pain relief may be provided independent of improvement of the disease or condition or the underlying cause of the disease or condition. For example, although the underlying disease may not improve, or may continue to progress, an individual suffering from the disease may experience pain relief. In some embodiments, enhanced bioavailability of the zoledronic acid may be achieved in treating one of these conditions by administering a dosage form comprising zoledronic acid in the form of a disodium salt. This may allow a reduced molar amount of the disodium salt to be used as compared to what would be used with the diacid form.

In some embodiments, the mammal being treated is not suffering from bone metastasis. In some embodiments, the mammal being treated is not suffering from cancer. In some embodiments, the mammal being treated is not suffering from osteoporosis.

For example, zoledronic acid or another bisphosphonate may be administered orally to relieve musculoskeletal pain including low back pain, and pain associated with rheumatoid arthritis, juvenile rheumatoid arthritis, osteoarthritis, erosive osteoarthritis, sero-negative (non-rheumatoid) arthropathies, non-articular rheumatism, peri-articular disorders, axial spondyloarthritis including ankylosing spondylitis, Paget's disease, fibrous dysplasia, SAPHO syndrome, transient osteoarthritis of the hip, vertebral crush fractures, osteoporosis, etc. In some embodiments, enhanced bioavailability of the zoledronic acid may be achieved in treating one of these conditions by administering a dosage form comprising zoledronic acid in the form of a disodium salt. This may allow a reduced molar amount of the disodium salt to be used as compared to what would be used with the diacid form.

A bisphosphonate, such as zoledronic acid, may also be used to treat bone fractures or to enhance the healing of bone fractures.

In some embodiments, zoledronic acid or another bisphosphonate may also be administered orally to relieve neuropathic pain, including diabetic peripheral neuropathy, post-herpetic neuralgia, trigeminal neuralgia, monoradiculopathies, phantom limb pain, and central pain. Other causes of neuropathic pain include cancer-related pain, lumbar nerve root compression, spinal cord injury, post-stroke pain, central multiple sclerosis pain, HIV-associated neuropathy, and radio-therapy or chemo-therapy associated neuropathy. In some embodiments, enhanced bioavailability of the zoledronic acid may be achieved in treating one of these conditions by administering a dosage form comprising zoledronic acid in the form of a disodium salt. This may allow a reduced molar amount of the disodium salt to be used as compared to what would be used with the diacid form.

In some embodiments, zoledronic acid or another bisphosphonate may be administered orally to relieve inflammatory pain including musculoskeletal pain, arthritis pain, and complex regional pain syndrome. In some embodiments, enhanced bioavailability of the zoledronic acid may be achieved in treating one of these conditions by administering a dosage form comprising zoledronic acid in the form of a disodium salt. This may allow a reduced molar amount of the disodium salt to be used as compared to what would be used with the diacid form.

Examples of musculoskeletal pain include low back pain; and pain associated with vertebral crush fractures, fibrous dysplasia, osteogenesis imperfecta, Paget's disease of bone, transient osteoporosis, and transient osteoporosis of the hip.

A bisphosphonate, such as zoledronic acid, may also be used to treat low back pain, or other musculoskeletal or inflammatory conditions, having a change in bone that is detectable by MRI or another medical imaging instrument. For example, a bisphosphonate, such as zoledronic acid, may be used to treat low back pain associated Modic changes, or vertebral endplate signal changes (VESC) and bone marrow changes visible using magnetic resonance imaging (MRI). Modic changes, can be classified into various types including type 1 (M1), type 2 (M2), and type 3 (M3) lesions or changes, any of which may be treated using a bisphosphonate such as zoledronic acid. VESCs may be found in patients with different types of low back pain including but not limited to spondylitis, trauma, spondyloarthropathies including ankylosing spondylitis, Schmorl's nodes, fracture, tumor, and spinal cord infarction. Lesions in ankylosing spondylitis include osteitis and spondylodiscitis which can be detected using MRI or another medical imaging instrument.

A bisphoshosphonate, such as zoledronic acid may also be used to treat osteoarthritis of the knee, such as osteoarthritis of the knee associated with bone marrow lesions (BML), including BML that may be detected using MRI or another medical imaging instrument. In some embodiments, a bisphosphonate, such as zoledronic acid, may be used to treat osteoarthritis of the knee associated with bone marrow edema (BME), including BME which may be detected using MRI or another medical imaging instrument.

Arthritis refers to inflammatory joint diseases that can be associated with pain. Examples of arthritis pain include pain associated with osteoarthritis, erosive osteoarthritis, rheumatoid arthritis, juvenile rheumatoid arthritis, sero-negative (non-rheumatoid) arthropathies, non-articular rheumatism, peri-articular disorders, neuropathic arthropaties including Charcot's foot, axial spondyloarthritis including ankylosing spondylitis, and SAPHO syndrome.

In some embodiments, a human being that is treated for a disease or condition, such as an inflammatory condition, e.g. arthritis, by an oral dosage form of zoledronic acid, has an age of about 10 years to about 90 years, about 20 years to about 80 years, about 30 years to about 75 years old, about 40 years to about 70 years, about 1 year to about 16 years, or about 80 years to about 95 years.

In some embodiments, a human being that is treated for a disease or condition, such as an inflammatory condition, e.g. arthritis, by an oral dosage form of zoledronic acid, has suffered from the arthritis for at least 1 month, at least 2 months, at least 6 months, or at least 1 year.

In some embodiments, the disease or condition, such as an inflammatory condition, e.g. arthritis, affects a knee, an elbow, a finger, a wrist, a shoulder, or a hip.

In some embodiments, zoledronic acid or another bisphosphonate may be administered orally to relieve complex regional pain syndrome, such as complex regional pain syndrome type I (CRPS-I), complex regional pain syndrome type II (CRPS-II), CRPS-NOS, or another type of CRPS. CRPS is a type of inflammatory pain. CRPS can also have a neuropathic component.

Complex regional pain syndrome is a debilitating pain syndrome. It is characterized by severe pain in a limb accompanied by edema, and autonomic, motor and sensory changes.

With respect to use of oral zoledronic acid for relieving pain associated with an inflammatory condition, relief of pain can be short-term, e.g. for a period of hours after administration of the dosage form, and/or relief of pain can be long-term, e.g. lasting for days, weeks, or even months after oral administration of zoledronic acid. In some embodiments, a mammal, such as a human being, experiences significant pain relief at least about 3 hours, at least about 6 hours, at least about 12 hours, at least about 24 hours, at least about 48 hours, at least about one week, at least about 2 weeks, or at least about 3 weeks after administration of an oral dosage form comprising zoledronic acid. In some embodiments, a mammal, such as a human being, experiences significant pain relief during at least part of the time from about 3 hours to about 2 weeks, about 3 hours to about 3 weeks, about 3 hours to about 24 hours, about 6 hours to about 2 weeks, or about 6 hours to about 24 hours, about 3 days to about 2 weeks, about 6 days to about 2 weeks, after administration of an oral dosage form comprising zoledronic acid.

Zoledronic acid or another bisphosphonate may also be administered orally to relieve cancer-related pain, including pain associated with multiple myeloma and bone metastases from solid tumors. In some embodiments, zoledronic acid is used to treat pain that is not cancer-related pain. For example, zoledronic acid may be used to treat pain that is not associated with multiple myeloma, bone metastasis from solid tumors, hypercalcemia of malignancy, giant cell tumor of bone, blood cancers or leukemias, or solid tumors or cancers.

In addition to relieving pain, oral administration of zoledronic acid or another bisphosphonate may also be useful to treat diseases or conditions that may or may not include a pain component. For example, zoledronic acid or another bisphosphonate may be useful to treat any of the pain conditions or types of conditions listed above, including treatment that does not simply relieve the pain of those conditions, and treatment that is carried out in such a way that the condition is treated without pain relief occurring. In addition to any pain relief zoledronic acid or another bisphosphonate may or may not provide, zoledronic acid or another bisphosphonates may be used to treat a disease or condition such as a metabolic disease or condition; an inflammatory disease or condition, including an inflammatory disease or condition that is not associated with pain; a cancer disease or condition; a neurological disease or condition; etc.

In some embodiments, oral administration of zoledronic acid or another bisphosphonate may also be useful to treat complex regional pain syndrome, rheumatoid arthritis, osteoarthritis, erosive osteoarthritis, axial spondyloarthritis including ankylosing spondylitis, acute vertebral crush fracture, fibrous dysplasia, SAPHO syndrome, osteoporosis, transient osteoporosis, or transient osteoporosis of the hip.

In some embodiments, oral administration of zoledronic acid or another bisphosphonate may also be useful to treat hypercalcemia of malignancy, multiple myeloma, bone metastases from solid tumors, Paget's disease of bone, giant cell tumor of bone, blood cancers or leukemias, or solid tumors or cancers.

In some embodiments, a bisphosphonate such as zoledronic acid may be used to treat otosclerosis.

Zoledronic acid, known as (1-hydroxy-2-imidazol-1-yl-1-phosphono-ethyl)phosphonic acid, is depicted by the following chemical structure:

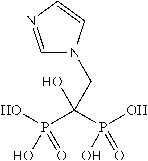 [SMILES: O=P(O)(O)C(O)(Cn1ccnc1)P(=O)(O)O]

In general, active pharmaceutical ingredients (APIs) in the pharmaceutical compositions can be prepared in a variety of different forms including prodrugs, amorphous forms, solvates, hydrates, cocrystals, salts and polymorphs.

Cocrystals, salts, solvates and hydrates of zoledronic acid could be used for medicinal purposes such as the treatment of pain. Of particular interest are compositions, cocrystals, or complexes comprising zoledronic acid and the standard amino acids since they might have enhanced permeability compared with other forms of zoledronic acid.

U.S. 20110028435, incorporated by reference herein for the compositions it describes, hypothesizes that zoledronic acid:amino acid complexes (a zoledronic acid:lysine complex and a zoledronic acid:glycine complex, have a structure shown below. The diagrams show a postulated molecular structure of the complex and possible interactions between the constituents of the complex which is different from the physical mix of the constituents.

1. Zoledronic Acid: Lysine Complex

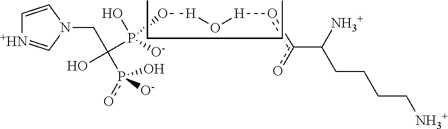

2. Zoledronic Acid: Glycine Complex

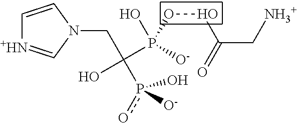

According to U.S. 20110028435, these represent one of the arrangements that molecules of zoledronic acid and the standard amino acids coformers could interact to form a stable complex that even when stressed thermally at elevated relative humidity (RH) environment have not displayed any signs of deterioration or disintegration to its original constituents. The inventors of U.S. 20110028435 believed that such stability can be attributed to the hydrogen bonding (dashed line in the box) in these molecular complexes. When packing in a crystal structure these complexes were reported to have very different morphologies to that of its constituents or their physical mix as indicated by their powder X-ray diffraction (PXRD) patterns and therefore would possess different, unpredictable physicochemical properties.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of a zoledronic acid, sodium zoledronate and water complex, characterized by an X-ray powder diffraction pattern having strong peaks at about 8.1, 13.3, 21.5, 24.6, and 25.6±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of an ammonium zoledronic salt and water complex, characterized by an X-ray powder diffraction pattern having strong peaks at about 11.0, 14.6, 15.4, 19.9, and 29.4±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of a zoledronic, L-lysine, and water complex, characterized by an X-ray powder diffraction pattern having strong peaks at about 9.0, 14.4, 18.1, 26.0, and 29.6±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of a zoledronic, DL-lysine, and water complex, characterized by an X-ray powder diffraction pattern having strong peaks at about 9.1, 14.7, 18.0, 21.2, and 26.0±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of a zoledronic acid, zoledronic, DL-lysine, ethanol, and water complex, characterized by an X-ray powder diffraction pattern having strong peaks at about 8.8, 9.7, 17.6, 23.1, and 26.5±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of a zoledronic acid, nicotinamide, and water complex, characterized by an X-ray powder diffraction pattern having strong peaks at about 13.1, 15.2, 21.0, 23.9, and 26.5±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of a zoledronic, adenine, and water complex, characterized by an X-ray powder diffraction pattern having strong peaks at about 13.6, 15.9, 19.7, 27.9, and 29.5±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of a zoledronic and glycine complex, characterized by an X-ray powder diffraction pattern having strong peaks at about 10.2, 17.8, 19.9, 22.9, and 28.1±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of a zoledronic diammonia water complex, characterized by an X-ray powder diffraction pattern having strong peaks at about 12.2, 13.0, 14.1, 17.1, and 19.3±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of a zoledronic, DL-lysine, and water complex, characterized by an X-ray powder diffraction pattern having strong peaks at about 8.3, 11.8, 12.3, 15.8, and 20.8±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of a zoledronic acid, L-lysine, and water complex, characterized by an X-ray powder diffraction pattern having strong peaks at about 9.6, 10.7, 14.3, 21.4, 23.5±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form comprises zoledronic acid in the form of a zoledronic, DL-lysine, and water complex, characterized by an X-ray powder diffraction pattern having strong peaks at about 9.7, 10.8, 14.4, 18.9, 21.4±0.2 degrees two-theta.

In some embodiments, a solid form of zoledronic acid may be used to treat a medical condition, (such as inflammatory pain, musculoskeletal pain, arthritis, low back pain, complex regional pain syndrome, etc.), wherein the solid form of zoledronic acid includes complexes of zoledronic acid with sodium, ammonium, ammonia, L-lysine, DL-lysine, nicotinamide, adenine and glycine which are capable of complexing in the solid-state, for example, through dry or solvent-drop grinding (liquid assisted grinding), heating or solvent evaporation of their solution in single or mixed solvent systems, slurry suspension, supercritical fluids or other techniques known to a person skilled in the art.

In some embodiments, a solid form of zoledronic acid may be used to treat a medical condition, (such as inflammatory pain, musculoskeletal pain, arthritis, low back pain, complex regional pain syndrome, etc.), wherein zoledronic and nicotinamide are complexed by dissolving both compounds in water:ethylacetate (1:1 v/v) and allowing the solvent mixtures to evaporate to form crystalline material.

In some embodiments, a solid form of zoledronic acid may be used to treat a medical condition, (such as inflammatory pain, musculoskeletal pain, arthritis, low back pain, complex regional pain syndrome, etc.), wherein a zoledronic and glycine solid complex is prepared by dissolving both compounds in water, and allowing the solvent to evaporate to form crystalline material.

Some embodiments include complexes of zoledronic acid and sodium, ammonium, ammonia, L-lysine, DL-lysine, nicotinamide, adenine and glycine suitable for a pharmaceutical formulation than can be delivered orally to the human body. The pharmaceutical formulation contains a therapeutically effective amount of at least one of the molecular complexes of zoledronic acid described herein and at least one pharmaceutically acceptable carrier, (also known in the art as a pharmaceutically acceptable excipient). The molecular complexes of zoledronic acid are therapeutically useful for the treatment and/or prevention of disease states associated with osteoporosis, hypercalcemia (TN), cancer induced bone metastasis, Paget's disease, adjuvant or neoadjuvant therapies, pain, or another condition discussed above.

Some embodiments include treating a medical condition, (such as inflammatory pain, musculoskeletal pain, arthritis, low back pain, complex regional pain syndrome, etc.), using molecular complexes of zoledronic acid referred to herein or a pharmaceutical formulation containing them. A pharmaceutical formulation may contain a molecular complex of zoledronic acid described herein. The pharmaceutical formulation may be, for example, a tablet, capsule, liquid suspension, injectable, suppository, topical, or transdermal. The pharmaceutical formulations generally contain about 1% to about 99% by weight of at least one molecular complex of zoledronic acid referred to herein and 99% to 1% by weight of a suitable pharmaceutical excipient.

U.S. 20110028435 reported that complexes of zoledronic acid and sodium, ammonium, ammonia, L-lysine, DL-lysine, nicotinamide, adenine, and glycine have been observed by their PXRD patterns and FTIR spectra.

In some embodiments, a solid form of zoledronic acid may be used to treat a medical condition, (such as inflammatory pain, musculoskeletal pain, arthritis, low back pain, complex regional pain syndrome, etc.), wherein preparation of the solid form of zoledronic acid includes the addition of excess at least one coformer to the zoledronic acid complexes, which may be the same as the coformer in the complex, a different coformer, or a mixture thereof.

In some embodiments, the excess cocrystal formers consist of standard amino acids.

In some embodiments, a solid form of zoledronic acid may be used to treat a medical condition, (such as inflammatory pain, musculoskeletal pain, arthritis, low back pain, complex regional pain syndrome, etc.), wherein the solid form is characterized by modified PK profiles of zoledronic acid complexes with excess cocrystal formers, compared with that of the orally delivered parent compound.

In some embodiments, a solid form of zoledronic acid may be used to treat a medical condition, (such as inflammatory pain, musculoskeletal pain, arthritis, low back pain, complex regional pain syndrome, etc.), wherein the solid form is characterized by improved aqueous solubility of zoledronic acid complexes compared with the parent compound.

In some embodiments, a solid form of zoledronic acid may be used to treat a medical condition, (such as inflammatory pain, musculoskeletal pain, arthritis, low back pain, complex regional pain syndrome, etc.), wherein the solid form is characterized by modified oral bioavailability values of zoledronic acid complexes with excess cocrystal formers, compared with the orally delivered parent compound.

In some embodiments, a solid form of zoledronic acid may be used to treat a medical condition, (such as inflammatory pain, musculoskeletal pain, arthritis, low back pain, complex regional pain syndrome, etc.), wherein the solid form is characterized by modified oral bioavailability values in dogs of zoledronic acid complexes prepared by the method referred to herein delivered in gelatin capsules compared with the orally delivered parent compound.

In some embodiments, a solid form of zoledronic acid may be used to treat a medical condition, (such as inflammatory pain, musculoskeletal pain, arthritis, low back pain, complex regional pain syndrome, etc.), wherein the solid form is characterized by modified oral bioavailability values in dogs of zoledronic acid complexes prepared by the method referred to herein delivered in enteric coated gel capsules compared with that of the parent compound.

In some embodiments, a solid form of zoledronic acid may be used to treat a medical condition, (such as inflammatory pain, musculoskeletal pain, arthritis, low back pain, complex regional pain syndrome, etc.), wherein the solid form is characterized by substantial improvement in oral bioavailability values in dogs of zoledronic acid complexes with excess cocrystal formers prepared by the method referred to herein delivered in hard gelatin capsules.

In some embodiments, a solid form of zoledronic acid may be used to treat a medical condition, (such as inflammatory pain, musculoskeletal pain, arthritis, low back pain, complex regional pain syndrome, etc.), wherein the solid form is characterized by improvement in oral bioavailability values for zoledronic acid in dogs via zoledronic acid and zoledronic acid complexes orally delivered through enteric coated capsules.

In some embodiments, a solid form of zoledronic acid may be used to treat a medical condition, (such as inflammatory pain, musculoskeletal pain, arthritis, low back pain, complex regional pain syndrome, etc.), wherein the solid form is characterized by a reduced oral bioavailability values for zoledronic acid in dogs via zoledronic acid complexes with excess physical mix of coformer.

In some embodiments, a solid form of zoledronic acid may be used to treat a medical condition, (such as inflammatory pain, musculoskeletal pain, arthritis, low back pain, complex regional pain syndrome, etc.), wherein the solid form is characterized by a molecular complex comprising a bisphosphonic acid or salt thereof and at least one coformer, wherein the bioavailability of the bisphosphonic acid or salt thereof from the molecular complex is greater than the bioavailability of the bisphosphonic acid or salt thereof without the coformer. The bisphosphonic acid may be, for example, zoledronic acid, clodronic acid, tiludronic acid, pamidronic acid, alendronic acid, residronic acid ibandronic acid or other bisphosphonic acids known in the art.

Another aspect provides a method for enhancing the bioavailabilty or permeability of a bisphosphonic acid to treat a medical condition, (such as inflammatory pain, musculoskeletal pain, arthritis, low back pain, complex regional pain syndrome, etc.), comprising the step of administering to a patient in need thereof a therapeutically effective of a bisphosphonic acid in the form of a molecular complex.

### EXAMPLES

Zoledronic acid as a starting material used in all experiments in this disclosure is supplied by Farmkemi Limited (Wuhan Pharma Chemical Co.), China with purity of ca. 98% and was purified further via recrystallization from water. All other pure chemicals (Analytical Grade) are supplied by Sigma-Aldrich and used without further purification.

Enteric coating of gelatin capsules: A 10% w/w coating solution of Eudragit L100-55, and triethyl citrate, 9.09 and 0.91 w/w % respectively, in purified water and acetone is used in the Vector LDCS pan coater to achieve a uniform coating layer on the capsules.

### Example 1

Preparation of Zoledronic Acid, Sodium Zoledronic Salt, and Water Complex

200 mg of zoledronic acid is slurried with 180 mg of sodium chloride in 1 mL of 1:1 ethanol:water overnight. The material is filtered and rinsed. The particulate material is gathered and stored.

### Example 2

Preparation of Ammonium Zoledronic Salt and Water Complex

300 mg of zoledronic acid is slurried in 7N ammonia in methanol overnight. The material is filtered and rinsed. The particulate material is dissolved in water and left to evaporate at ambient conditions to obtain colorless plates after 1 week.

### Example 3

Preparation of Zoledronic, L-Lysine, and Water Complex

200 mg of zoledronic acid and 54 mg of L-lysine are slurried in 2 mL of tetrahydrofuran and 200 μl of water overnight. The solids gathered after filtration are dried and stored.

### Example 4

Preparation of Zoledronic, DL-Lysine, and Water Complex

204 mg of zoledronic acid and 59 mg of DL-lysine are slurried in 2 mL of tetrahydrofuran and 200 μl of water overnight. The solids gathered after filtration are dried and stored.

### Example 5

Preparation of Zoledronic Acid, Zoledronic, DL-Lysine, Ethanol, and Water Complex

103 mg of zoledronic acid and 54 mg of DL-lysine are dissolved in 400 μA of water, capped and stirred overnight. The next day 0.25 mL of ethanol is added drop wise. The vial is capped with a screw cap vial and after 1 day crystals appear and are filtered off. The material is stored.

### Example 6

Preparation of Zoledronic, Nicotinamide, and Water Complex by Solvent-Drop Grinding

99 mg of zoledronic acid is ground with 44 mg of nicotinamide and 40 μl of water is added to the solid mixture. The solids gathered after grinding are stored.

### Example 7

Preparation of Zoledronic, Nicotinamide, and Water Complex from Solution Crystallization

25 mg of zoledronic acid and 138 mg of nicotinamide are dissolved in 2 mL of a water:ethylacetate mix (1:1 v/v). The solution is then allowed to stand for several hours to effect the slow evaporation of solvent. The solids are gathered.

### Example 8

Preparation of Zoledronic, Adenine, and Water Complex by Solvent-Drop Grinding

96 mg of zoledronic acid is ground with 65 mg of adenine and 60 μL of water is added to the solid mixture. The solids gathered after grinding are stored.

### Example 9

Preparation of Zoledronic, Adenine, and Water Complex from Solution Slurry

99 mg of zoledronic acid and 54 mg of adenine are slurried in 2 mL of a water:ethanol mix (1:1 v/v) overnight. The solids gathered after filtration are dried.

### Example 10

Preparation of Zoledronic and Glycine Complex

178 mg of zoledronic acid and 45 mg of glycine are slurried in 2 mL of water overnight. The solids gathered after filtration are dried and stored.

### Example 11

Preparation of Zoledronic Diammonia Water Complex

1.5 g of zoledronic acid is slurried in 7N ammonia in methanol overnight. The material is filtered and rinsed. The particulate material is dissolved in water with medium heat and left to evaporate at ambient conditions to obtain colorless blocks after 1 day.

### Example 12

Preparation of Zoledronic, DL-Lysine, and Water Complex

200 mg of zoledronic acid and 102 mg of DL-lysine are slurried in 2 mL of tetrahydrofuran and 400 μl of water overnight. The solids gathered after filtration are dried and stored.

### Example 13

Preparation of Zoledronic, DL-Lysine, and Water Complex

1 g of zoledronic acid and 283 mg of DL-lysine are slurried in 80 mL of tetrahydrofuran and 8 mL of water overnight. The solids gathered after filtration are dried and stored.

### Example 14

Preparation of Zoledronic, DL-Lysine, and Water Complex by Antisolvent Method

This complex can also be prepared by the antisolvent method by dissolving 1 g of zoledronic acid and 283 mg of DL-lysine in 5 mL of hot water and adding 40 mL of ethanol as an antisolvent stirred overnight.

### Example 15

Preparation of Zoledronic, L-Lysine, and Water Complex

1 g of zoledronic acid and 255 mg of L-lysine are dissolved in 60 mL of hot water. 100 mL of ethanol is then added as an antisolvent. The solids gathered after filtration are dried and stored.

### Example 16

The following oral dosage form may be prepared.

| | Active Ingredient | | Coformer |
|---|---|---|---|
| Compound | Dose | | Amount |
| Zoledronic Acid in PEG400 | 5 | mg/kg | n/a |
| Zoledronic Acid, sodium zoledronic salt, and | 5 | mg/kg | n/a |
| water complex in PEG400 | | | |
| Zoledronic Acid and glycine complex in | 5 | mg/kg | n/a |
| PEG400 | | | |
| Zoledronic Acid, nicodinamide, and water in | 5 | mg/kg | n/a |
| PEG400 | | | |
| Zoledronic Acid, L-lysine, and water complex | 5 | mg/kg | n/a |
| in PEG400 | | | |
| Zoledronic Acid, DL-lysine and water | 5 | mg/kg | n/a |
| complex in PEG400 | | | |
| Zoledronic Acid | 5 | mg/kg | n/a |
| Zoledronic Acid and glycine complex | 5 | mg/kg | n/a |
| Zoledronic Acid, DL-lysine and water | 5 | mg/kg | n/a |
| complex | | | |
| Zoledronic Acid, L-lysine, and water complex | 5 | mg/kg | n/a |
| Zoledronic acid and glycine complex, solid | 5 | mg/kg | 45 mg/kg |
| suspension in PEG400 | | | glycine |
| Zoledronic acid and glycine complex, solid | 5 | mg/kg | 25 mg/kg |
| suspension in PEG400 | | | glycine |
| Zoledronic acid and glycine complex, solid | 5 | mg/kg | 5 mg/kg glycine |
| suspension in PEG401 | | | |
| Zoledronic acid and DL-lysine, and water | 5 | mg/kg | 39.32 mg/kg |
| complex, solid suspension in PEG400 | | | DL-lysine |
| Zoledronic acid and DL-lysine, and water | 5 | mg/kg | 28.8 mg/kg DL- |
| complex, solid suspension in PEG401 | | | lysine |
| Zoledronic acid and DL-lysine, and water | 5 | mg/kg | 5.62 mg/kg DL- |
| complex, solid suspension in PEG402 | | | lysine |
| Zoledronic acid and DL-lysine, and water | 5 | mg/kg | n/a |
| complex, solid suspension in PEG403 | | | |
| Zoledronic Acid monohydrate | 56.0 mg; enteric | | n/a |
| | coated capsules | | |
| Zoledronic Acid and glycine complex | 67.0 mg; enteric | | n/a |
| | coated capsules | | |
| Zoledronic Acid, DL-lysine and water | 87.7 | mg | 294.8 mg/kg |
| complex | | | DL-lysine |
| Zoledronic Acid, DL-lysine and water | 87.7 mg; enteric | | 294.8 mg/kg |
| complex | coated capsules | | DL-lysine |
| Zoledronic Acid, DL-lysine and water | 84.2 | mg | 294.8 mg/kg |
| complex | | | DL-lysine |
| Zoledronic Acid, DL-lysine and water | 87.7 mg; enteric | | n/a |
| complex | coated capsules | | |
| Zoledronic Acid, DL-lysine and water | 35.4 | mg | 123.8 mg/kg |
| complex | | | DL-lysine |
| Zoledronic Acid, DL-lysine and water | 35.4 | mg | 294.8 mg/kg |
| complex | | | DL-lysine |
| Zoledronic Acid and glycine complex | 67.0 | mg | 294.8 mg/kg |
| | | | glycine |
| Zoledronic Acid, L-lysine, and water complex | 87.7 | mg | 294.8 mg/kg |
| | | | L-lysine |
| Zoledronic Acid | 0.183 | mg/kg | n/a |
| Zoledronic Acid | 0.12 | mg/kg | n/a |

### Embodiments

The following embodiments are contemplated.

Embodiment 1. A method of treating pain, comprising administering, to a mammal in need thereof, a molecular complex comprising a bisphosphonic acid or salt thereof and at least one coformer, wherein the bioavailability of the bisphosphonic acid or salt thereof from the molecular complex is greater than the bioavailability of the bisphosphonic acid or salt thereof without the coformer.

Embodiment 2. A method of treating pain, comprising administering, to a mammal in need thereof, a molecular complex of embodiment 1, wherein the bisphosphonic acid is selected from the group consisting of zoledronic acid, clodronic acid, tiludronic acid, pamidronic acid, alendronic acid, residronic acid and ibandronic acid.

Embodiment 3. A method of treating pain, comprising administering, to a mammal in need thereof, a molecular complex of embodiment 1, wherein at least one coformer is an amino acid.

Embodiment 4. A method of treating pain, comprising administering, to a mammal in need thereof, a molecular complex of embodiment 1, wherein the bisphosphonic acid is zoledronic acid and at least one coformer is an amino acid.

Embodiment 5. A method of treating pain, comprising administering, to a mammal in need thereof, a molecular complex of embodiment 1, wherein at least one coformer is lysine.

Embodiment 6. A method of treating pain, comprising administering, to a mammal in need thereof, a composition comprising a molecular complex of embodiment 1 and an excess amount of at least one coformer.

Embodiment 7. A method of treating pain, comprising administering, to a mammal in need thereof, a composition of embodiment 6, wherein the excess coformer is present in an amount up 100.times. the mass of the molecular complex.

Embodiment 8. A method of treating pain, comprising administering, to a mammal in need thereof, a pharmaceutical composition comprising a composition of embodiment 7 and a pharmaceutically acceptable excipient.

Embodiment 9. A method of treating pain, comprising administering, to a mammal in need thereof, a pharmaceutical composition comprising a composition of embodiment 6 and a pharmaceutically acceptable excipient.

Embodiment 10. A method of treating pain, comprising administering, to a mammal in need thereof, a pharmaceutical composition of embodiment 8 wherein the pharmaceutical composition is an oral dosage form.

Embodiment 11. A method of treating pain, comprising administering, to a mammal in need thereof, a pharmaceutical composition of embodiment 9, wherein the pharmaceutical composition is an oral dosage form.

Embodiment 12. A method of treating pain, comprising administering, to a mammal in need thereof, a molecular complex of embodiment 1, wherein the molecular complex is crystalline.

Embodiment 13. A method for enhancing the bioavailabilty or permeability of a bisphosphonic acid or salt thereof for the treatment of pain comprising the step of administering to a patient in need thereof a therapeutically effective amount of a bisphosphonic acid in the form of a molecular complex according to embodiment 1.

Embodiment 14. A method for enhancing the bioavailabilty or permeability of a bisphosphonic acid or salt thereof comprising the step of administering to a patient in need thereof a therapeutically effective amount of a bisphosphonic acid in the form of a composition according of embodiment 5.

Embodiment 15. A method for enhancing the bioavailabilty or permeability of a bisphosphonic acid or salt thereof comprising the step of administering to a patient in need thereof a therapeutically effective amount of a bisphosphonic acid in the form of a composition according of embodiment 6.

Embodiment 16. A method for the treatment and/or prevention of disease states associated with osteoporosis, hypercalcemia, cancer induced bone metastasis, Paget's disease or adjuvant or neoadjuvant cancer therapies comprising the step of administering to a patient in need thereof a therapeutically effective amount of a bisphosphonic acid or salt thereof in the form of a composition according of embodiment 5.

Embodiment 17. A method for the treatment and/or prevention of disease states associated with osteoporosis, hypercalcemia, cancer induced bone metastasis, Paget's disease or adjuvant or neoadjuvant cancer therapies comprising the step of administering to a patient in need thereof a therapeutically effective amount of a bisphosphonic acid or salt thereof in the form of a composition according of embodiment 6.

Embodiment 18. A method of treating pain, comprising administering, to a mammal in need thereof, a crystalline form of zoledronic acid comprising zoledronic acid, water, and a compound selected from L-lysine; DL-lysine; nicotinamide; adenine; and a zoledronic acid salt.

Embodiment 19. A method of treating pain, comprising administering, to a mammal in need thereof, a crystalline form of zoledronic acid according to embodiment 18, wherein the crystalline form isa crystalline zoledronic acid, sodium zoledronate and water complex characterized by an X-ray powder diffraction pattern having peaks at about 8.1, 13.3, 21.5, 24.6, and 25.6±0.2 degrees two-theta;a crystalline ammonium zoledronic acid salt and water complex characterized by an X-ray powder diffraction pattern having strong peaks at about 11.0, 14.6, 15.4, 19.9, and 29.4±0.2 degrees two-theta;a zoledronic diammonia water complex characterized by an X-ray powder diffraction pattern having strong peaks at about 12.2, 13.0, 14.1, 17.1, and 19.3±0.2 degrees two-theta;a crystalline zoledronic acid, L-lysine, and water complex characterized by an X-ray powder diffraction pattern having peaks at about 9.0, 14.4, 18.1, 26.0, and 29.6±0.2 degrees two-theta;a crystalline zoledronic acid, L-lysine, and water complex characterized by an X-ray powder diffraction pattern having peaks at about 9.6, 10.7, 14.3, 21.4, 23.5±0.2 degrees two-theta;a crystalline zoledronic acid DL-lysine and water complex characterized by an X-ray powder diffraction pattern having peaks at about 8.3, 11.8, 12.3, 15.8, and 20.8±0.2 degrees two-theta;a crystalline zoledronic acid, DL-lysine, and water complex characterized by an X-ray powder diffraction pattern having peaks at about 9.1, 14.7, 18.0, 21.2, and 26.0±0.2 degrees two-theta;a crystalline zoledronic acid, DL-lysine, and water complex characterized by an X-ray powder diffraction pattern having peaks at about 9.7, 10.8, 14.4, 18.9, 21.4±0.2 degrees two-theta;a crystalline zoledronic acid, zoledronic, DL-lysine, ethanol, and water complex characterized by an X-ray powder diffraction pattern having peaks at about 8.8, 9.7, 17.6, 23.1, and 26.5±0.2 degrees two-theta;a crystalline zoledronic acid, adenine, and water complex characterized by an X-ray powder diffraction pattern having peaks at about 13.6, 15.9, 19.7, 27.9, and 29.5±0.2 degrees two-theta; ora crystalline zoledronic acid, nicotinamide, and water complex characterized by an X-ray powder diffraction pattern having strong peaks at about 13.1, 15.2, 21.0, 23.9, and 26.5±0.2 degrees two-theta.

Embodiment 20. A method of treating pain, comprising administering, to a mammal in need thereof, a crystalline form of zoledronic acid comprising zoledronic acid and glycine.

Embodiment 21. A method of treating pain, comprising administering, to a mammal in need thereof, a molecular complex of zoledronic acid comprising zoledronic acid and glycine.

Embodiment 22. A method of treating pain, comprising administering, to a mammal in need thereof, a crystalline form of zoledronic acid according to embodiment 20, wherein the crystalline form is a crystalline zoledronic acid and glycine complex characterized by an X-ray powder diffraction pattern having peaks at about 10.2, 17.8, 19.9, 22.9, and 28.1±0.2 degrees two-theta.

Embodiment 23. A method of treating pain, comprising administering, to a mammal in need thereof, a molecular complex of zoledronic acid comprising zoledronic acid, water, and a compound selected from L-lysine; D,L-lysine; nicotinamide; adenine; and a zoledronic acid salt or comprising zoledronic acid and glycine.

Embodiment 24. A method of treating pain, comprising administering, to a mammal in need thereof, a molecular complex of zoledronic acid according to embodiment 23 selected from the group consisting of: a zoledronic acid, sodium zoledronate and water complex,an ammonium zoledronic acid salt and water complex,a zoledronic diammonia water complex,a zoledronic acid, L-lysine, and water complex,a zoledronic acid DL-lysine and water complex,a zoledronic acid, zoledronic, DL-lysine, ethanol, and water complex,a zoledronic acid, adenine, and water complex,a zoledronic acid, nicotinamide, and water complex, or a zoledronic acid glycine complex.

Embodiment 25. A method of treating pain, comprising administering, to a mammal in need thereof, a molecular complex comprising zoledronic acid and lysine.

Embodiment 26. A method of treating pain, comprising administering, to a mammal in need thereof, a crystalline form comprising zoledronic acid and lysine.

Embodiment 27. A method of treating pain, comprising administering, to a mammal in need thereof, a pharmaceutical composition comprising a complex of embodiment 18 and a pharmaceutically acceptable excipient.

Embodiment 28. A method of treating pain, comprising administering, to a mammal in need thereof, a pharmaceutical composition according to embodiment 27, wherein the composition is a oral solid dosage form.

Embodiment 29. A method for the treatment and/or prevention of disease states associated with osteoporosis, hypercalcemia, cancer induced bone metastasis, Paget's disease or adjuvant or neoadjuvant cancer therapies comprising the step of administering to a patient in need thereof a therapeutically effective amount of a pharmaceutical composition according to embodiment 27.

Embodiment 30. A method of treating pain, comprising administering a composition or molecular complex of any preceding embodiment to a mammal in need thereof.

Embodiment 31. The method of embodiment 30, wherein the pain is arthritis pain.

Embodiment 32. The method of embodiment 30, wherein the pain is inflammatory pain.

Embodiment 33. The method of embodiment 30, wherein the pain is associated with complex regional pain syndrome.

Embodiment 34. The method of embodiment 30, wherein the pain is low back pain.

Embodiment 35. The method of embodiment 30, 31, 32, 33, or 34, wherein the bisphosphonate is zoledronic acid.

1. A method of treating pain, comprising administering, to a mammal in need thereof, a molecular complex comprising zoledronic acid and lysine, wherein the molecular complex is a solid.

2. The method of claim 1, wherein the molecular complex is administered in a pharmaceutical composition further comprising a pharmaceutically acceptable excipient.

3. The method of claim 2, wherein the composition is an oral solid dosage form.

4. The method of claim 3, wherein the composition is an oral dosage form selected from a tablet and a capsule.

5. The method of claim 2 wherein the pharmaceutical composition further comprises an excess amount of lysine.

6. The method of claim 1, wherein the molecular complex is crystalline.

7. The method of claim 7, wherein the molecular complex is in a pharmaceutical composition that further comprises a pharmaceutically acceptable excipient.

8. The method of claim 7, wherein the composition is an oral solid dosage form.

9. The method of claim 8, wherein the composition is an oral dosage form selected from a tablet and a capsule.

10. The method of claim 6, wherein the molecular complex is administered in a pharmaceutical composition that further comprises an excess amount of lysine.

11. A method of treating pain, comprising administering, to a mammal in need thereof, a molecular complex comprising zoledronic acid or a salt thereof and lysine, wherein the molecular complex is a solid and the bioavailability of the zoledronic acid or salt thereof from the molecular complex is greater than the bioavailability of the zoledronic acid or salt thereof without lysine.

12. The method of claim 11, wherein the molecular complex is in a pharmaceutical composition that further comprises a pharmaceutically acceptable excipient.

13. The method of claim 12, wherein the composition is an oral solid dosage form.

14. The method of claim 13, wherein the composition is an oral dosage form selected from a tablet and a capsule.

15. The method of claim 11, wherein the molecular complex is in a composition that further comprises an excess amount of lysine.

16. The method of claim 11, wherein the molecular complex is crystalline.

17. The method of claim 16, wherein the molecular complex is in a composition that further comprises a pharmaceutically acceptable excipient.

18. The method claim 17, wherein the composition is an oral solid dosage form.

19. The method of claim 18, wherein the composition is an oral dosage form selected from a tablet and a capsule.

20. The method of claim 16, wherein the molecular complex is in a composition that further comprises an excess amount of lysine.

21. The method of claim 1, wherein the pain is arthritis pain.

22. The method of claim 1, wherein the pain is inflammatory pain.

23. The method of claim 1, wherein the pain is associated with complex regional pain syndrome.

24. The method of claim 1, wherein the pain is low back pain.

25. The method of claim 11, wherein the pain is arthritis pain.

26. The method of claim 11, wherein the pain is inflammatory pain.

27. The method of claim 11, wherein the pain is associated with complex regional pain syndrome.

28. The method of claim 11, wherein the pain is low back pain.
